(19) United States
(12) Patent Application Publication
(10) Pub. No.: US 2026/0335107 A1
(43) Pub. Date: Oct. 29, 2026

(54) Bonded Prosthetic Liner

(71) Applicant: Alps South, LLC, St. Petersburg, FL (US)

(72) Inventors: Aldo Laghi, Pinellas Park, FL (US); David Carson, Clearwater, FL (US); Jakob W McClelland, Gulfport, FL (US); Nathaniel Vint, Palm Harbor, FL (US)

(21) Appl. No.: 19/731,455

(22) Filed: Jul. 6, 2026

Related U.S. Application Data
(62) Division of application No. 18/661,095, filed on May 10, 2024.
(60) Provisional application No. 63/467,365, filed on May 18, 2023.
(60) Provisional application No. 63/524,329, filed on Jun. 30, 2023.

(51) Int. Cl. A61F 2/78

(52) U.S. Cl. CPC A61F 2/7812

(57) ABSTRACT

A prosthetic liner having a cylindrical or conical body with an open proximal end, a closed distal end, a posterior face, an anterior face, an internal thermoplastic elastomeric layer, and an external fabric layer, with a support material attached to the anterior face and a plurality of interior surface modifications on the internal thermoplastic elastomeric layer with the option of including at least one channel for wicking. The liner may be a locking or cushion liner. Also, the support material may be attached to the distal end of the liner inside of an enclosed pocket or inside of an open pocket attached to the exterior fabric layer. The support material is preferably a foam the foam selected from the group consisting of ethyl vinyl acetate, polyethylene, neoprene, ethylene propylene diene terpolymer, vinyl, silicone, and urethane. These liners allow for a bulbous formation at the distal end of a residual limb to give comfort to a user who may have a traumatic injury, scar tissue buildup, or simply seeking further comfort.

### CROSS-REFERENCE TO RELATED APPLICATIONS

This application is a divisional application of pending application No. Ser. No. 18/661,095, filed May 10, 2024 which claimed priority to provisional Application No. 63/467,365, filed May 18, 2023, and application No. 63/524,329, filed Jun. 30, 2023, the disclosures of which are incorporated in their entirety by reference herein.

### BACKGROUND OF THE INVENTION

### Field of the Invention

The described invention relates to prosthetic liners. Specifically, the described invention relates to a prosthetic liner having varied supporting layers.

### Description of the Background Art

Prosthetic liners have been in use since the 1970's, mostly custom made, and made of various materials. Prosthetic liners serve the purpose of providing comfort for an amputee when wearing a prosthetic limb as the liner allows for a cushion between the residual limb and the socket supporting the prosthetic limb. Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Pat. No. 5,507,834 to Laghi et al., U.S. Pat. No. 5,443,525 to Laghi et al., and U.S. Pat. No. 5,728,168 to Laghi et al. Gel and urethane liners have also been used for prosthetic and orthotic purposes and, for the most part, have a fabric covering. The fabric covering is used to reinforce the underlying material (silicone, gel, urethane) and allows for extensibility to make it easier to don and doff the liner by rolling it on and off the residual limb.

The liners disclosed in U.S. Pat. Nos. 5,443,525, 5,507,234, 5,728,168, 6,544,292, and 6,764,631 each represent advances in the field of fabric covered liners and the disclosures of which are hereby incorporated by reference. Additionally, U.S. Pat. No. 6,454,812, also hereby incorporated by reference, describes a liner comprising additional features attached to the liner textile to limit the vertical stretch at the distal end of a liner and is incorporated herein by reference. This method has been used successfully within the field of prosthetics to provide the liner textile with a strengthened distal end, means for attaching a threaded mechanical feature, and a boundary layer that prevents the thermoplastic elastomer from undesirably passing through the textile during high pressure processing.

The issue with each of these prior liners is that they lack the ability to incorporate padding, bladders, or other supporting material. This is because liners, as they are currently manufactured, either require injection molding or compression molding to bond the thermoplastic elastomeric layer to the exterior fabric layer. These processes generally require some form of pressure that is too great for supporting material to be reliably applied as the pressure causes the support material to deform.

Therefore, it is an object of this invention to provide an improvement which overcomes the aforementioned inadequacies of the prior art devices and provides an improvement which is a significant contribution to the advancement of the reinforced prosthetic liner art.

Another object of the invention is to provide a prosthetic liner formed using a textured mandrill creating surface modification of the interior thermoplastic elastomeric layer to allow for the addition of pads and other materials that would otherwise melt or become improperly configured due to the heat and/or pressure involved in standard molding processes.

Another object of the invention is to provide a prosthetic liner that allows for the inclusion of foam pads, spacer fabric, air bladders, fluid bladders, and other support materials.

Another object of the invention is to provide a prosthetic liner that allows for the introduction of electrical relays and sensors.

Another object of the invention is to provide a prosthetic liner that forms a composite material using heat and/or chemical bonding.

Another object of the invention is to provide a prosthetic that includes internal channels in the interior thermoplastic elastomeric layer that allows for the drainage of moisture.

Another object of the invention is to provide a prosthetic liner that includes therapeutic agents embedded in the interior thermoplastic elastomeric layer.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a prosthetic liner having a cylindrical or conical body with an open proximal end, a closed distal end, a posterior face, an anterior face, an internal thermoplastic elastomeric layer, and an external fabric layer, with a support material attached to the anterior face and a plurality of interior surface modifications on the internal thermoplastic elastomeric layer with the option of including at least one channel for wicking. The liner may be a locking or cushion liner. Also, the support material may be attached to the distal end of the liner inside of an enclosed pocket or inside of an open pocket attached to the exterior fabric layer. The support material is preferably a foam the foam selected from the group consisting of ethyl vinyl acetate, polyethylene, neoprene, ethylene propylene diene terpolymer, vinyl, silicone, and urethane. These liners allow for a bulbous formation at the distal end of a residual limb to give comfort to a user who may have a traumatic injury, scar tissue buildup, or simply seeking further comfort.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a front perspective view of an embodiment of the present invention;

FIG. 2 is a cross-sectional view of the liner shown in FIG. 1 along line J-J;

FIG. 3 is a front perspective view of an alternative embodiment of the present invention;

FIG. 4 is a cross-sectional view of the liner shown in FIG. 3 along line L-L;

FIG. 5 is a cross-sectional view of the present invention with FIG. 5A showing a portion of the inner wall of the liner from element Q;

FIG. 6 is a front perspective view of an embodiment of the present invention with a support material adhered to the exterior fabric layer;

FIG. 7 is a cross-sectional view of the liner shown in FIG. 6 along line H-H;

FIG. 8 is a front perspective view of another embodiment of the present invention with a support material included as part of a removable pocket;

FIG. 9 is a cross-sectional view of the liner shown in FIG. 8 along line I-I;

FIG. 10 is a front perspective view of another embodiment of the present invention with a support material partially enclosed in a pocket;

FIG. 11 is a cross-sectional view of the liner shown in FIG. 10 along line K-K;

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

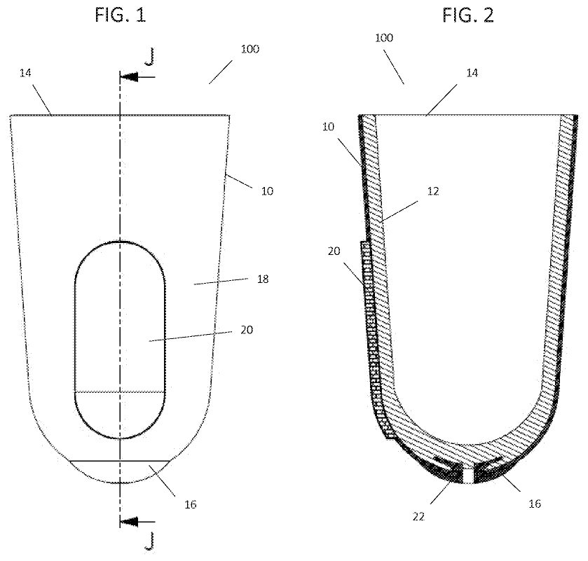

As shown in FIG. 1 and FIG. 2, one embodiment of the liner 100 is shown. The liner 100 generally has an external fabric layer 10 and an internal thermoplastic elastomeric layer 12 with an open proximal end 14 and a closed distal end 16. The internal thermoplastic elastomeric layer 12 is preferably a styrene-based thermoplastic elastomer such as SEEPS but may be any thermoplastic and elastomeric material that is heat-formable and comfortable on a user's skin. The anterior face 18 of liner 100 includes a support material 20. Support material 20 can be adhered or sewn onto or into the external fabric layer 10 prior to bonding with the internal thermoplastic elastomeric layer 12 and can be a foam, spacer fabric (i.e. a multi-layered fabric with internal stitching allowing for a spring-like structure), an air bladder, a fluid bladder, or some other padded material that increases patient comfort. If adhered, a urethane-based adhesive is used on the external fabric layer 10 to prevent the fabric/support material combination from moving when the external fabric layer is being attached to the internal thermoplastic layer 12. The support material 20 is preferably sized in relation to the size of the liner 100 and is preferably about ½ the width and ⅔ the height of the liner 100 but may be sized according to patient need. As seen in more detail in FIG. 2, one of embodiment of the liner 100 can also include a distal lock 22 which are common in prosthetic liners and known in the art.

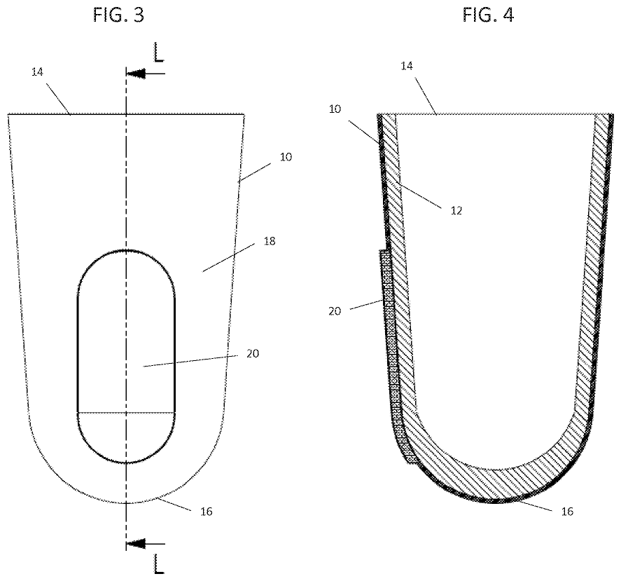

As shown in FIG. 3 and FIG. 4, another embodiment of the liner 100 does not need the distal lock 22 but is rather a suction-based liner. The liner 100 still incorporates an open proximal end 14, a closed distal end 16, an internal thermoplastic elastomeric layer 12, an external fabric layer 10, and a support material 20. For this particular embodiment, the support material 20 can also be placed on the distal end 16 as opposed to the anterior face 18.

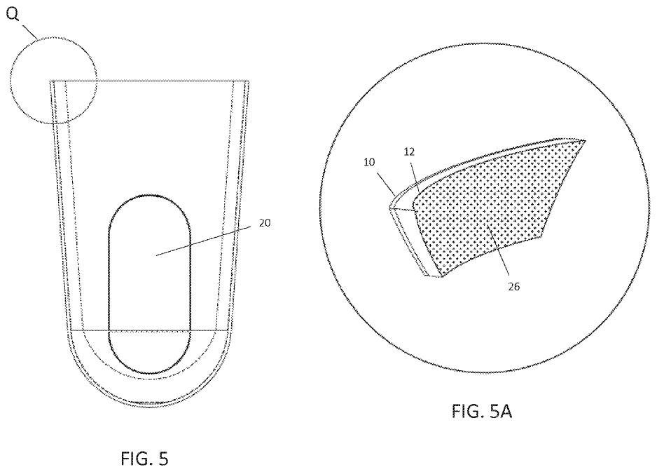

The internal surface 24 of the internal thermoplastic elastomeric layer 12 has interior surface modifications 26, as seen in FIG. 5A. The interior surface modifications 26 are useful by allowing the surface to grip a user's skin better thereby preventing movement and skin breakdown. The interior surface modifications 26 are effectively dimples throughout the internal thermoplastic elastomeric layer 12. Alternatively, the interior surface modifications 26 can include internal channels to allow moisture to drain down the surface to the distal end 16 (which may include a moisture port) or to wick upward towards the open proximal end 14. These internal channels may also be used for air which may become trapped during the donning procedure. The internal surface 24 may have different shapes or textures to create different effects. In addition, the internal surface 24 could have therapeutic agents such as antioxidant creams, antimicrobial agents, or pain management agents applied. This serves the benefit of having a known amount of whatever agent is used being applied to a user's skin as opposed to the potentially unknown amount caused by having the agent incorporated into the full thickness of the internal thermoplastic elastomeric layer 12.

The present invention can also incorporate electrical arrays or load bearing sensors that would normally be damaged through a typical molding and forming process. Likewise, sealing prosthetic liners can be formed by placing seals under the external fabric layer 12 to form a ring for sealing. In another embodiment the internal thermoplastic elastomeric layer 12 could comprise a softer styrene-based gel on a posterior side and a denser styrene-based gel on the anterior side to protect the tibial crest. This allows for the creation of liners that have varying beneficial characteristics related to stretch, elongation, and general comfort.

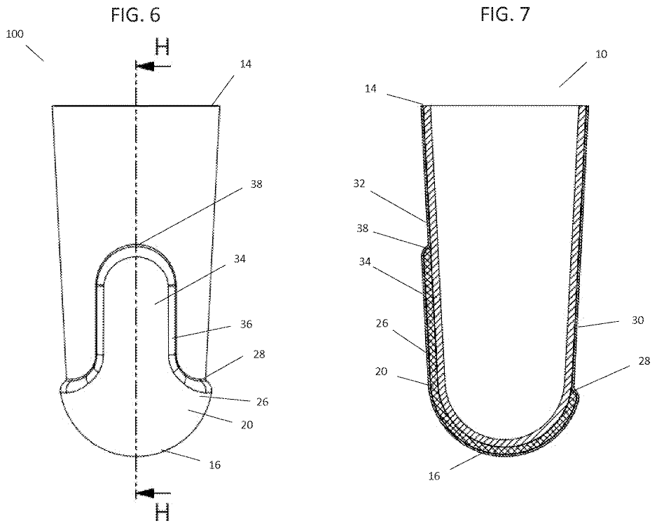

In situations where foam is used for the support layer 20, the support layer foam 26 can be open or closed-cell and should be compressible. As seen in FIGS. 6 and 7, support layer foam 26 is preferably placed distally because amputees having bony protrusions or nerve sensitivity will encounter those issues in those areas of the liner. Similarly, having padding distally is beneficial for amputees with high loading forces (i.e. high activity such as running, jumping, etc.) The support layer foam 26 can be made of a variety of materials such as ethyl vinyl acetate (EVA), polyethylene, an ionomer, neoprene, ethylene propylene diene terpolymer (EPDM), vinyl, silicone, or urethane. The support layer foam 26 may be cut into a variety of shapes. For example, as seen in FIGS. 6 and 7, the support layer foam 26 may circumferentially surround the closed distal end 16 which tapers to a circumferential end point 28 on the posterior side 30 of the liner 10 whereas on the anterior side 32 the support layer foam 26 extends upwardly towards the open proximal end 14 forming an anterior protrusion 34 which terminates at an anterior end point 38 which is prior to the open proximal end 14 and preferably about half of the length of the liner 10. However, it should be understood by those of ordinary skill in the art that a variety of shapes and configurations can be pursued based on patient need. The support layer foam 26 in this embodiment further includes a tapered edge 36 that allows the support layer foam 26 to have a thickness that ranges from ½ an inch at its thickest point to ⅛th of an inch at its narrowest.

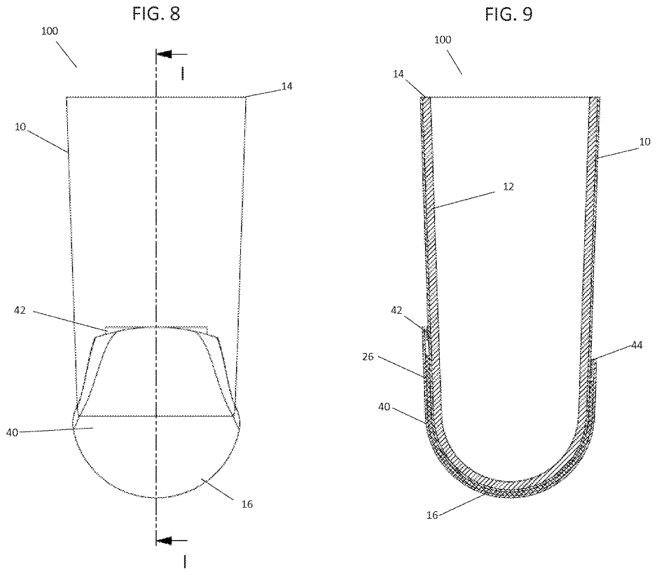

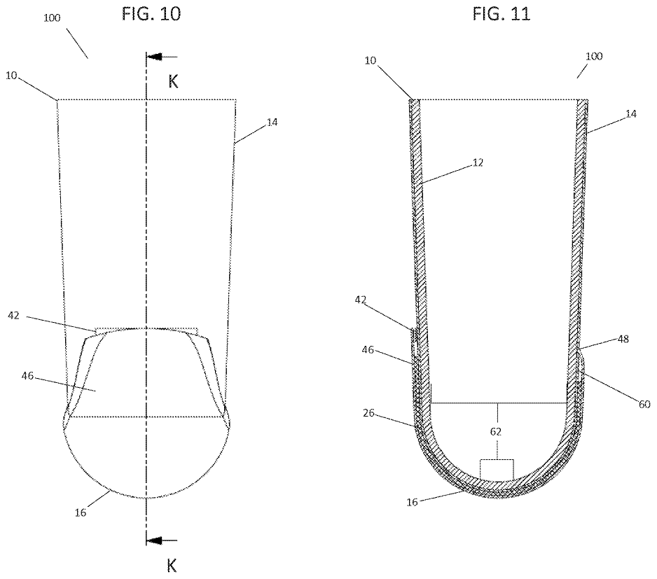

Alternatively, instead of having the support layer foam 26 included in the exterior fabric layer 10 prior to molding, the exterior fabric layer 10 of liner 100 may have an additional pocket added the exterior fabric layer 10 prior to molding over the internal thermoplastic elastomeric layer 12 as described above. As seen in FIG. 8 and FIG. 9, liner 100 may include a removable pocket 40 which is secured to the distal end 16 by an anterior hook and loop connection 42 and a posterior hook and loop connection 44. This setup allows the patient to add additional support layer foam 26 due to volume fluctuation or other comfort purposes or remove support layer foam 26 for similar reasons. Alternatively, as shown in FIG. 10 and FIG. 11, the support layer foam 26 can be inserted into a closed pocket 46 that is secured by an anterior hook and loop connection 42 and a closed anterior end 48. The support layer foam 26 does not necessarily have to fill the entirety of the cavity 60 formed by the closed pocket 46 when inserted into the closed pocket 46 but it should at least support the entire the curved region 62 of closed distal end 16.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A bonded prosthetic liner comprising:
a body with an open proximal end, a closed distal end, a posterior face, an anterior face, an internal thermoplastic elastomer layer, and an external fabric layer, wherein a support material is attached to the anterior face; and
a plurality of interior surface modifications on the internal thermoplastic layer.

2. The bonded prosthetic liner of claim 1 wherein the support material is chosen from the group consisting of foam, spacer fabric, an air bladder, a fluid bladder, and a padded material.

3. The bonded prosthetic liner of claim 1 wherein the cylindrical body further comprises a distal attachment.

4. The bonded prosthetic liner of claim 1 wherein the cylindrical body has a body width and a body height and wherein the support material has a material width about half of the body width and a material height of about ⅔ of the body height.

5. The bonded prosthetic liner of claim 1 further comprising at least one therapeutic agent incorporated into the internal thermoplastic layer.

6. The bonded prosthetic liner of claim 1 wherein the body is conical.

7. The bonded prosthetic liner of claim 1 wherein the body is cylindrical.

8. A bonded prosthetic liner comprising:
a body with an open proximal end, a closed distal end, a posterior face, an anterior face, an internal thermoplastic elastomer layer, and an external fabric layer, wherein a support material is attached to the closed distal end; and
a plurality of interior surface modifications on the internal thermoplastic layer.

9. The bonded prosthetic liner of claim 8 wherein the support material is chosen from the group consisting of foam, spacer fabric, an air bladder, a fluid bladder, and a padded material.

10. The bonded prosthetic liner of claim 8 wherein the cylindrical body has a body width and a body height and wherein the support material has a material width about half of the body width and a material height of about ⅔ of the body height.

11. The bonded prosthetic liner of claim 8 further comprising at least one therapeutic agent incorporated into the internal thermoplastic layer.

12. The bonded prosthetic liner of claim 8 wherein the body is conical.

13. The bonded prosthetic liner of claim 8 wherein the body is cylindrical.
